(19) United States
(12) Patent Application Publication
(10) Pub. No.: US 2011/0270407 A1
(43) Pub. Date: Nov. 3, 2011

(54) MEDICAL OR VETERINARY MATERIAL, METHOD FOR THE PRODUCTION AND USE THEREOF

(75) Inventor: Jean-Pierre COUGOULIC, Pornichet (FR)

(73) Assignee: CADOREL, CATHERINE, LA BAULE (FR)

(21) Appl. No.: 13/015,667

(22) Filed: Jan. 28, 2011

Related U.S. Application Data
(63) Continuation-in-part of application No. 10/540,756, filed on Jun. 24, 2005, now Pat. No. 7,902,270.

(30) Foreign Application Priority Data
Dec. 24, 2002 (FR) 02/ 16627

(51) Int. Cl. A61F 2/28

(52) U.S. Cl. 623/23.61

(57) ABSTRACT

A sterile endosseous implant suitable for an insertion into a living tissue, the implant includes a moulded piecework made of poly (etheretherketon) as a binder and the moulded piecework includes an external graft surface embedded crystallized calcium phosphate particles emerging from the surface.

### CROSS REFERENCES TO RELATED APPLICATIONS

This application is a continuation-in-part of co-pending application Ser. No. 10/540,756 filed on Jun. 24, 2005, which is the 35 U.S.C. §371 national stage of International PCT/FR03/50208 filed on Dec. 23, 2003, which claims priority to French Application No. 02/16627 filed on Dec. 24, 2002. The entire contents of each of the above-identified applications are hereby incorporated by reference.

### BACKGROUND OF THE INVENTION

1. Field of the Invention

The present invention refers to an endosseous implant, with enhanced osseo-integration characteristics which may be used in the medical or veterinary field, in particular but not exclusively for dental implants, or for bone prostheses.

2. Related Art

Numerous types of materials, metal or plastic materials are used in the medical or veterinary field for replacing biological structures (bone in particular) or for fastening functional organs (dental implants or others . . . ).

The material is selected in relation to its intrinsic structural characteristics and also in relation to its biocompatibility in terms of tolerance or, even better, in terms of biological acceptance.

The document FR-A-2722694 describes a moulded material for the realisation of endo-bone implants or of bone prostheses, made of thermoplastic polymer (in particular poly(etheretherketone), also called PEEK) including calcium hydroxyapatite, tricalcic phosphate, orthophosphoric acid and a TiO₂-type zeolite.

Being manufactured by a moulding process, this related art implant is suitable for an economic mass production. However, in spite of the encouraging results obtained with this type of material, it appears that the results in terms of biological integration are not quite satisfactory.

### BRIEF SUMMARY OF THE INVENTION

In order to overcome the shortcomings of the related art, the invention relates to a sterile endosseous implant suitable for an insertion into a living tissue, the implant including:
a moulded piecework made of poly(etheretherketon) as a binder;
the moulded piecework including an external graft surface including embedded crystallized calcium phosphate particles emerging from the surface.

According to an advantageous embodiment the graft surface also includes zeolite particles the particles being not covered by the binder.

Preferably these zeolite particles are made of TiO₂.

According to preferred embodiment the graft surface is in a semi crystallized state.

### BRIEF DESCRIPTION OF VIEWS OF THE DRAWINGS

The invention will now be described according to its preferred embodiment with reference to the FIGS. 1 to 4 where:

FIG. 1 is a diagram of the manufacturing process of an implant according to the invention

FIG. 2 is a magnified view of a longitudinal cross section of the surface of an implant according to the invention, showing the evolution of this surface according to the manufacturing steps of the implant;

FIG. 3 shows an embodiment example of a dental implant;

FIG. 4 is a comparison of the cell colonization rate of the surface of an implant according to the invention compared with a reference sample.

### DETAILED DESCRIPTION

According to the current invention the implant is in the form of a moulded part made of a biocompatible binder containing one or several compounds for adding calcium and phosphorus, which moulded part exhibits a specific surface condition that increases the cell colonization rate of the surface, the surface condition being provided through an economic and reproducible surface pickling process.

The combination of surface roughness and surface distribution of active compound, in particular calcium and phosphorus, promotes the creation of ionic links between such added elements and the surrounding chemical elements, mineral or organic elements, after biological implantation of the part.

These active compounds are resorptive once inserted into a living tissue, such as a bone, and further promote through this resorption process the mechanical and chemical sealing of the implant in the tissue.

The biocompatible binder is selected in relation to its physical characteristics after shaping in particular by an injection-moulding operation. By way of example, a thermoplastic polymer such as poly(etheretherketon), ketone polyether, amide block polyether, polytétrafluoréthylène or still polyimide may be used; a natural polymer, in particular such as cellulose, may also be used.

Because of its high Young's modulus and its interesting structural characteristics, close to those of the bone, poly(etheretherketone) (PEEK) is used preferably. PEEK is a semi-crystalline polymer made of an aromatic linear chain

The characteristics of this polymer are expanded on in the commercial leaflet published in 1992 by ICI MATERIALS: <<Victrex PEEK, the high temperature engineering thermoplastic-properties and processing>>.

The additions of calcium and phosphorus are composed advantageously of calcium phosphates derived form example from tricalcic phosphate (Ca₃(PO₄)₂), dicalcic or monetite phosphate (CaHPO₄), with stoichiometric formulation ((Ca₃(PO₄)₃OH) or (Ca10(PO₄)₆H₂O)), with stoichiometric formulation or not, or of products containing the elements.

The presence of calcium phosphates enables the material to approximate the natural composition of the bone in order to enhance the biocompatibility thereof. Products containing calcium phosphates, which are at least partially resorptive, are used preferably.

In particular, calcium hydroxyapatite is a component that can be found in the bone. It can be used advantageously in its non-stoichiometric form, since it is then slightly resorptive, which is interesting for cellular integration.

Dicalcic or tricalcic phosphate is advantageously cheap and one of the basic biological components for the formation of calcium hydroxyapatite; it is also resorptive and has also a healing function.

These various additions of calcium phosphates may also be used as mixtures.

Besides the addition of calcium phosphates, the implant according to the invention can be made of a material containing orthophosphoric acid (H₃PO₄). Natural orthophosphoric acid is prescribed as a calcium fixative and as an acidifier; it is also a fundamental component of the nucleotides which as the basic units of nucleic acids, which partake of the constitution of the nucleus of living cells. Moreover, the material is advantageously laden with one or several compounds enabling to create or promote the electrostatic links with the surrounding medium. This(these) charge(s) may be selected among zeolites and/or certain oxides: using ceramics such as titanium dioxide (TiO₂), zirconium dioxide (ZrO₂), aluminum oxide (Al₂0₃) or silicon dioxide (SiO₂) may be contemplated.

The charges in question are electrostatic compounds which allow ionic bonding function; they have moreover high molar mass and they contribute to improve the radio-opacity of the material.

FIG. 3 is an example of a dental implant 300. Such an implant extends over a longitudinal axis 301. As known form the related art, such an implant exhibits advantageously macroscopic serrations or raised patterns 310 for its mechanical anchoring into the tissue, e.g., a bone tissue. These macroscopic patterns make the inserted part of the implant behaves like a plug into the hole where it is inserted. The dimension of this macroscopic pattern is in the millimeters range. The invention focuses on the surface condition at a microscopic level, i.e. a surface condition that can only be revealed through scanning electron microscope observations.

FIG. 2 outlines the microscopic scale conditions on a detailed, highly magnified view of the implant surface seen in a longitudinal cross section.

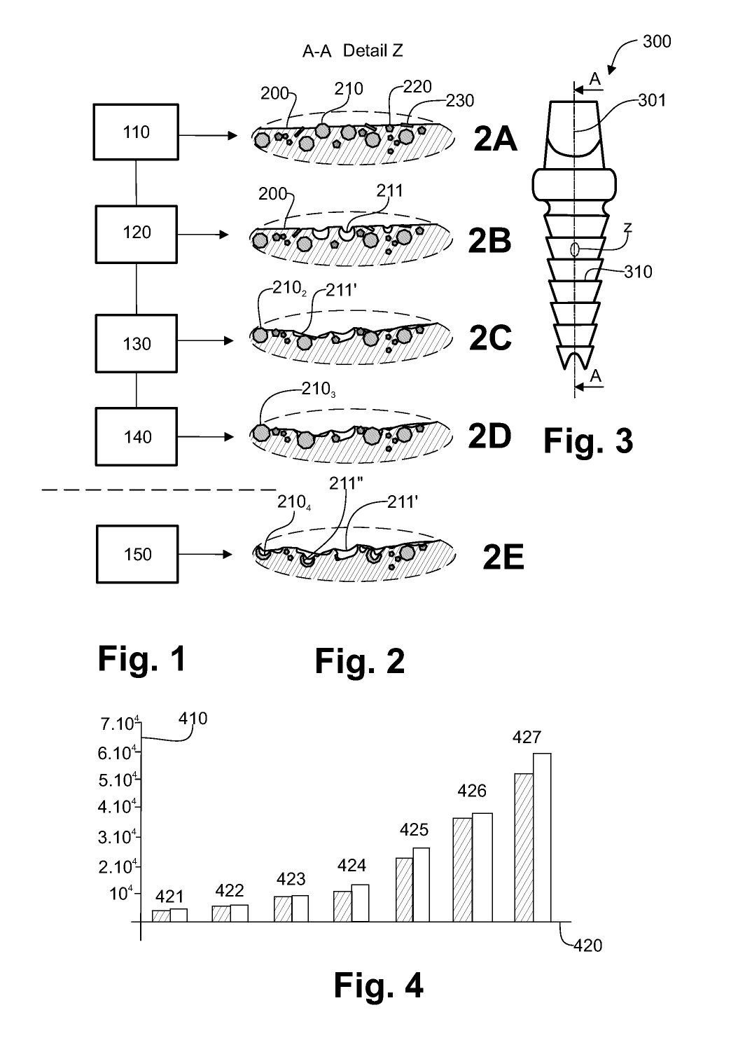

Going now to FIG. 1, the first manufacturing step 110 consists in a moulding operation of a compound including the above mentioned constituents.

To keep a mouldable material with sufficient handling and resistance, the polymer binder represents at least 65%, and preferably 65% to 90%, in weight of the part.

On the other hand, to add sufficient quantity of chemical elements intended to promote the biological integration, the complementary components (tricalcic phosphate and/or dicalcic phosphate and/or calcium hydroxyapatite, possibly associated with at least one compound of zeolite or oxide type for example, intended to improve electrostaticity and radio-opacity, and with orthophosphoric acid) represent between 10 and 35% in weight of the material making the part.

A good compromise, in particular in terms of mechanical characteristics corresponds substantially to 80% in weight of polymer binder and 20% in weight of complementary component(s).

When looking at the surface condition in FIG. 2A, the surface 200 is essentially smooth as a result of the high moulding pressure, the material being pressed against the mould walls, and may exhibit, in some places, some emerging particles made of calcium phosphate 210 or zeolite 220. However the surface 200 is also contaminated by various particles 230 like metallic particles originated e.g. from micro chipping of the part of the injection machine or from the mold. Therefore the implant is not suitable for an insertion into the living tissue of a patient at this stage.

Because of the contamination of the surface, the surface must be forcefully decontaminated, by performing a surface decontamination step 120 where the implant is soaked into an acid bath, such as hydrochloric or sulphuric acid, the bath being subjected to ultrasounds.

Going to FIG. 2B, this surface decontamination treatment results in the dissolution of the metallic particles 230 but also of the calcium phosphate particles that where emerging from the surface 200, or that were not covered by a sealed layer of binder, the binder being not dissolved by the acid. As a result of this selective chemical attack, the surface 200 is left with cavities 211 at the places where metallic or calcium phosphate particles were laying. The surface roughness increases accordingly.

After rinsing in a bath of water subjected to ultrasounds in order to remove any acid segregation form the surface 200, the implant is subjected to a soak into acetone the bath being subjected to ultrasounds in order to perform a surface layer decontamination step 130. This step 130 removes a layer of binder as this layer might be partially contaminated or be in an amorphous state because of a thermal quenching of the material contacting the colder walls of the mould during the injection process. This acetone bath removes this less dense layer but does not affect calcium phosphate or zeolite particles unless such particles are entirely included in the removed layer of binder. As a result, FIG. 2C, the surface 200 exhibits further cavities 211′ with emerging particles 210₂ either from the surface and also from the bottom of those cavities.

After rinsing a further step 140 consists in a sterilization which is performed by soaking the implant in hydrogen peroxide (H₂O₂, at 110 vol. or 30% for example), and/or sodium hypochloride (NaClO) used preferably in combination. Advantageously, complementary product baths are used with purely disinfecting function, such as GIGASEPT (registered trademark) or LYSETOL (registered trademark), all these bath being subjected to ultrasounds. Finally, the implant is inserted in a sterilization sheath for passing in an autoclave; it is then subjected to a sterilization cycle at high temperature and under a pressure. This sterilization operation by autoclave contributes to the surface pickling function.

As shown in FIG. 2D, the surface condition after the sterilization step, as a result of the autoclave treatment, the emerging calcium phosphate particles 210₃ crystallize.

The implant is now ready for insertion in to a bone tissue and can be conditioned in a sealed packaging.

Step 150 of FIG. 1 corresponds to the insertion of the implant into a living tissue and FIG. 2E gives an insight into the surface condition of the implant after a few weeks spent in this tissue.

The emerging particles of zeolite and calcium phosphate cooperate with the pre-existing cavities 211′ to promote the cell colonization of the surface 200. As the calcium phosphate particles are at least partially resorptive, the resorption process creates micro-cavities 211″ which are also colonized by cells.

FIG. 4 is an example of the evolution of cell colonization of the surface of an implant according to the invention having received the specific pickling treatment and exhibit the previously described features compared with a control sample consisting of poly (etherthercetone). The diagram shows the number of cells per cm² 410 measured on the surface with regard to time 420. Results are given for 3 hours 421, 6 hours 422, 1 day 423, 3 days 424, 9 days 425, 15 days 426 and 27 days 427. The control sample results are given by the hatched histograms while the results found on the material surface according to the invention are given by white histograms. These results show that the presence of particles at the surface of the implant promotes the cell colonization from the first hours of insertion into the tissue.

### EXAMPLES

Basic mixtures are prepared out of poly(etheretherketone) (PEEK), tricalcic phosphate (Ca₃(PO₄)₂), and titanium dioxide (TiO₂).

The PEEK is in the form of a powder or of granules (size: approx. 100 microns), available from Victrex Europa GmbH, Hauptstr. 11 D-65719 HOFHEIM—Germany.

Tricalcic phosphate is available in the form of a powder (grain size close to 200 microns); it is for instance marketed by Cooperation Pharmaceutique Française, 77020 MELUN—France. Titanium oxide is also available in the form of a powder distributed by Cooperation Pharmaceutique Française, 77020 MELUN—France.

### a) Proportions

Some possible examples of compositions are specified below:

| Mixture 1 (10% charges) | Mixture 2 (20% charges) |
|---|---|
| PEEK 90% in weight | PEEK 80% in weight |
| Ca₃(PO₄)₂: 5% in weight | Ca₃(PO₄)₂ 10% in weight |
| TiO₂: 5% in weight | TiO₂: 10% in weight |
| Mixture 3 (30% charges) | Mixture 4 |
| PEEK 70% in weight | PEEK: 65% in weight |
| Ca₃(PO₄)₂: 15% in weight | Ca₃(PO₄)₂: 17.5% in weight |
| TiO₂: 15% in weight | TiO₂: 7.5% in weight |

### b) Mingling

The constituents of each mixture are placed in a turbine mixer until perfect homogenising.

### c) Drying

Each homogeneous mixture obtained is dried in an air circulation stove for 3 hours at 150° C.

### d) Moulding

The moulding operation is performed on a KRAUSS-MAFFEL-type injection press. Model 90-340-32, KRAUSS MAFFEI FRANCE, 92632 GENNEVILLIERS—FRANCE.

The preparation conditions of the material and the moulding conditions of the mixture correspond to the commercial leaflet <<ICI MATERIALS>>, specified above.

PEEK being a semi-crystalline thermoplastic, it is necessary to heat the mould to a temperature at least greater than that of its vitreous transition (140° C.). Failing which the surface quality of the moulded parts would be affected. Indeed, the surface web would be in amorphous phase and the core in crystalline phase; if the mould were too cold, the parts might even have totally amorphous character and the mechanical characteristics would drop considerably.

Thermoregulation of the mould is ensured by an oil re-heater enabling to maintain it at a temperature of the order of 160° C. Insulation means limit thermal dispersions and preserve the peripheral organs of the injection press. Such means may be in the form of insulating plates formed of a fibre glass complex.

For series injections, a vibrator will be advantageously fixed to the hopper to promote the flow of the mixture.

Generally speaking, moulding is conducted at a temperature of the order of 340 to 400° C. and at an injection pressure close to 70 to 140 MPa.

The mould may be shaped in relation to the part to be obtained, for example for realising bone prosthesis, in particular for orthopaedic applications. A block of matter can also be obtained that will then be cut or machined to the desired shape, for bone filling or an implant, of dental type for example.

### e) Surface Pickling—Decontamination

After obtaining the moulded material, the former is subjected to surface pickling and decontamination operations, before aseptic conditioning.

The products used for these surface pickling and decontamination operations may be hydrochloric acid, (HCl, for example 30%) or sulphuric acid (H₂SO₄, for example 30%), acetone (C₃H₆O), hydrogen peroxide (H₂O₂, at 110 vol. or 30% for example), and/or sodium hypochloride (NaClO) used preferably in combination. Advantageously, complementary product baths are used with purely disinfecting function, such as GIGASEPT (registered trademark) or LYSETOL (registered trademark).

HCl 30%: 20 minutes

H₂O: 10 minutes (or rinsing)

acetone: 20 minutes

H₂O: 10 minutes (or rinsing)

H₂O₂ 30%: 20 minutes

NaClO: 20 minutes

H₂O: 10 minutes (or rinsing)

GIGASEPT 12%: 60 minutes

H₂O Ppi: 20 minutes (or rinsing)

The implant is inserted in a sterilisation sheath for passing in an autoclave; it is then subjected to a sterilisation cycle at a temperature of the order of 135° C. for 10 minutes, under a pressure of the order of 2150 mbars.

### f) Results

An electronic scanning microscope analysis shows that the pickling/decontamination and sterilisation operations promote the apparition of calcium phosphates in surface. These calcium phosphates emerge through micropores and crystallize.

After implantation, surface analysis shows the presence of holes and chaps at the surface of the material, and also the presence of carbon, oxygen and nitrogen, whereas little calcium and phosphorus can be found relative to the initial integrated concentrations.

This tends to show partial disappearance of the calcium phosphate particles in surface, and the colonisation of the holes and chaps by surrounding biological materials, sign of a graft-type biological acceptance.

Clinical analysis from inserted implants shows that the material in question develops at the contact thereof a cortical bone further to the physical and atomic characteristics of the material.

It is here a true graft principle; these results demonstrate the clinical reality of an integration of the material to the surrounding tissue.

What is claimed is:

1. A sterile endosseous implant suitable for an insertion into a living tissue, said implant comprising:
a moulded piecework made of poly (etheretherketon) as a binder;
said moulded piecework comprising an external graft surface including embedded crystallized calcium phosphate particles emerging from said surface.

2. An implant according to claim 1 wherein the graft surface also comprises zeolite particles said particles not being covered by the binder.

3. An implant according to claim 2, wherein zeolite particles are made of TiO₂.

4. An implant according to claim 1, wherein the graft surface is in a semicrystalline state.
